SEQUENCE LISTING (3 sequences)

SEQ ID NO: 1 - RNA, 22 nt, JC polyomavirus
  source 1..22: /organism=“JC polyomavirus” /mol_type=“unassigned RNA”
uucugagaccugggaaaagcau

SEQ ID NO: 2 - RNA, 22 nt, JC polyomavirus
  source 1..22: /organism=“JC polyomavirus” /mol_type=“unassigned RNA”
uucugagaccugggaagagcau

SEQ ID NO: 3 - RNA, 22 nt, BK polyomavirus
  source 1..22: /organism=“BK polyomavirus” /mol_type=“unassigned RNA”
aucugagacuugggaagagcau

(19) United States
(12) Patent Application Publication
(10) Pub. No.: US 2017/0218466 A1
(43) Pub. Date: Aug. 3, 2017

(54) DETECTION OF CIRCULATING JC POLYOMAVIRUS (JCPYV) MICRORNAS AS BIOMARKER FOR JCPYV INFECTION

(71) Applicant: Janssen Pharmaceutica NV, Beerse (BE)

(72) Inventors: Ole Siegfrid Lagatie, Beerse (BE); Lieven Jozef Stuyver, Beerse (BE)

(21) Appl. No.: 15/500,534

(22) PCT Filed: Jul. 30, 2015
(86) PCT No.: PCT/EP2015/067471

§ 371 Date: Jan. 31, 2017

(30) Foreign Application Priority Data
Jul. 31, 2014 (EP) 14179290.3

(51) Int. Cl. C12Q 1/70

(52) U.S. Cl. CPC C12Q 1/701; C12Q 2600/178; C12Q 2600/158

(57) ABSTRACT

The invention concerns the quantitative analysis of circulating JCPyV microRNAs (miRNA) in body fluid and its use as biomarker for JCPyV infection.

The invention relates to the quantitative analysis of circulating JCPyV microRNAs (miRNA) in body fluid.

Infection with JC Polyomavirus (JCPyV) is thought to occur early in childhood and it is estimated that 50-80% of the human population has been exposed to this virus as they are seropositive for the VP1 protein, which is the major constituent of the viral capsid.

The human JC polyomavirus (JCPyV) is the etiological agent of Progressive Multifocal Leukoencephalopathy (PML), a demyelinating disease of the brain caused by lytic infection of oligodendrocytes upon viral reactivation. JCPyV is a circular double-stranded DNA virus with very restricted cellular tropism, infecting oligodendrocytes, astrocytes, kidney epithelial cells and peripheral blood cells. It is thought that infection usually occurs asymptomatically in childhood, after which the virus remains latent in the body. Under certain immunocompromising conditions, such as treatment with immunomodulatory drugs (e.g. natalizumab) or infection with Human Immunodeficiency Virus (HIV), the virus can be reactivated and actively replicate into the brain, leading to PML. Current risk assessment for development of PML is mainly based on the detection of antibodies against VP1, the major capsid protein and the detection of viral DNA in urine (viruria). It has been reported that 50 to 80% of humans are seropositive for JCPyV and approximately one fifth of the population sheds JCPyV in the urine. Detection of viral DNA in plasma (viremia) is very rare and has been shown not to be useful for predicting PML risk. Recently it was shown however that viral DNA can be detected in CD34⁺ or CD19⁺ cells, with an increased detection rate in Multiple Sclerosis (MS) patients treated with natalizumab. As the risk of developing PML increases upon prolonged use of natalizumab, current treatment guidelines recommend discontinuation of therapy after the second year, particularly in JCPyV seropositive patients. Given the high prevalence of JCPyV antibodies, a large number of patients are advised to discontinue therapy. Although most, if not all, PML patients are seropositive or show seroconversion before diagnosis of PML, the overall incidence of PML in natalizumab-treated MS patients is not more than 1.1% in the highest risk group, indicating that not all seropositive subjects have the same risk of developing PML. Moreover, the introduction of a risk stratification algorithm, predominantly based on JCPyV serology, has not led to a reduction of PML incidence in natalizumab-treated MS patients. Therefore, development of new tools for improved risk stratification is highly needed, as this might justify continued therapy for many MS patients and better identify those patients who are really at risk of developing PML.

MicroRNAs (miRNAs) are small, non-coding RNAs that play an important role in fine-tuning the expression of specific gene products through translational repression and/or mRNA degradation and as such are implicated in many diseases. Cellular miRNAs can also be released in small vesicles, such as exosomes and the levels of these extracellular miRNAs in biological fluids have become very valuable markers of several diseases, such as cancer, Alzheimer's disease and diabetes. In the context of JCPyV, it was shown that there does not appear to be a relationship between circulating human miRNAs and the presence of anti-VP1 antibodies or urinary viral load.

Several viruses encode their own sets of miRNAs, which can have self-regulatory or host modulating roles. Also JCPyV, as well as other polyomaviruses, encodes its own unique microRNA that is produced as part of the late transcript in an infected host cell. These microRNAs are thought to play an important role in controlling viral replication through downregulation of Large T-Antigen expression, but also in controlling NKG2D-mediated killing of virus-infected cells by natural killer (NK) cells through downregulation of the stress-induced ligand ULBP3. The diagnostic potential of circulating viral miRNAs has already been investigated for Epstein-Barr virus and Kaposi's sarcoma-associated herpesvirus (KSHV), where they might represent potential markers for virus associated malignancies. Also JCPyV miRNA has been shown recently to be a useful biomarker for JCPyV infection in the gastrointestinal tract.

The current invention relates to the detection of JCPyV-encoded miRNAs in plasma or urine of healthy subjects and whether the presence of these miRNAs is related to VP1 serology or urinary viral load. It has been demonstrated that these viral miRNAs can indeed surprisingly be detected in plasma or urine and that they might be useful markers for viral activity.

Our analysis of the plasma or urine levels of JCPyV microRNAs has shown that more than 50% of healthy subjects do have detectable levels of JCPyV microRNAs thereby indicating these individuals are infected by JCPyV. Remarkably, this detection of viral miRNA is independent of the individual's serostatus or whether viral particles are detected in urine, thereby offering a novel approach for assessing the viral status of a subject and giving a more direct measure of the viral activity.

The invention comprises the quantitative analysis of plasma levels of viral microRNAs, more in particular JCV-miR-J1-5p and JCV-miR-J1a-5p by PCR-based technologies.

In a cohort of 50 healthy subjects it was investigated whether jcv-miR-J1-5p (and its variant jcv-miR-J1a-5p) can be detected in plasma or urine of healthy subjects. It was found that the overall detection rate of JCPyV miRNA was 74% (37/50) in plasma and 62% (31/50) in urine. Subjects were further categorized based on JCPyV VP1 serology status and viral shedding. In subjects that are JCPyV seronegative a detection rate of 86% (12/14) and 57% (8/14) was found in plasma and urine, respectively. In subjects that are JCPyV seropositive a detection rate of 69% (25/36) and 64% (23/36) was found in plasma and urine, respectively. Furthermore, in seropositive subjects shedding virus in urine, higher levels of urinary viral miRNAs were observed, when compared to seropositive subjects not shedding virus in urine (P<0.001). No correlation was observed between levels of urinary and plasma miRNAs. Taken together, the data indicate that analysis of plasma or urinary viral miRNAs divulge the presence of latent

JCPyV infection allowing further stratification of seropositive individuals. Also, these data indicate higher infection rates than would be expected from serology alone.

The current invention relates to a method to analyze the presence and/or activity of JCPyV in a subject by quantitatively assessing the levels of circulating JCPyV miRNAs within a sample derived from said subject by detecting said circulating JCPyV miRNAs comprising the steps of:
i. Extracting circulating JCPyV miRNAs from a sample derived from a subject and producing JCPyV miRNA containing miRNA extract;
ii. Reverse transcribing said JCPyV miRNA containing miRNA extract and producing JCPyV miRNA cDNA;
iii. Amplifying said JCPyV miRNA cDNA and forming a plurality of amplified JCPyV miRNA cDNAs and a plurality of complementary JCPyV miRNA cDNAs; and
iv. Detecting the presence and/or quantifying said plurality of amplified JCPyV miRNA cDNAs or said plurality of complementary JCPyV miRNA cDNAs and detecting and/or quantifying said circulating JCPyV miRNAs in said sample.

The method according to the invention also relates to detecting and quantifying said circulating JCPyV miRNAs in said sample identifying a JCPyV-infected subject subsequently.
In addition the invention relates to the method wherein detection and quantification comprises:
i. Determining an amount of said plurality of amplified JCPyV miRNA cDNAs or said plurality of complementary JCPyV miRNA cDNAs;
ii. Determining a level of circulating JCPyV miRNAs within said sample based on said amount; and
iii. Comparing said level to a standard control level, wherein said level being higher than said standard control level indicative of said subject developing or having PML.

The “subject” in the invention means a mammalian subject, a human subject or preferably an immunocompromised patient such as HIV-infected individuals or patients treated with immunomodulatory drugs e.g. natalizumab.

Said sample is either a fluid sample, like blood or urine, blood plasma or blood serum.

Although JCPyV is known to be the etiological agent responsible for development of PML, it is also well-known that this polyomavirus is widely distributed in the human population without any clinical manifestation. The level of the viral miRNAs in plasma and urine of healthy subjects has now been analyzed. As is the case for other polyomaviruses, JCPyV encodes a pre-miRNA that is further processed into the mature 5p and 3p miR-J1s. The pre-miRNA is encoded on the late strand of the viral genome and is shown to be expressed concurrently with the late mRNA transcript, thereby downregulating early transcript. The presence of viral miRNAs might therefore be considered as a marker for latent infection. Since BKPyV and JCPyV share the same 3p miRNA, only the 5p miRNAs were included in this study.

Most of the healthy subjects have low but well-detectable levels of viral miRNAs in total RNA extracts of plasma or urine, even in subjects that are seronegative and as such considered not to be infected. This finding indicates that the analysis of antibodies against JCPyV VP1 is insufficient to identify infected individuals. Previous studies also identified individuals that were seronegative but clearly infected based on the fact that they were viremic in specific cell compartments (CD34⁺ cells) or plasma. Whereas in most cases only a limited number of seronegative subjects were found to be infected, our analysis of viral miRNAs in plasma and urine identified, respectively 12 and 8 out of 14 seronegative individuals to be infected with JCPyV. Only 1 seronegative subject was found to be negative for both plasma and urine viral miRNAs. These data suggest that JCPyV is capable of evading immune recognition by the host's humoral immune system and residing latently in the body, with viral miRNAs leaking in the blood or urine as a silent witness of this latent activity.

Similar to the findings in seronegative subjects, also a large group of seropositive subjects was found to have viral miRNAs in plasma or urine. 69% of seropositive subjects were positive for plasma viral miRNAs and 64% were found to be positive for urinary viral miRNAs. Given the fact that viral miRNAs can only be produced upon ongoing viral transcription in infected host cells, whereas serology rather discloses information on exposure to the virus and more in particular the response of the immune system towards this exposure, the analysis of viral miRNAs in seropositive subjects might add another level to the risk prediction algorithms for PML development. Continued treatment with immunomodulatory drugs in subjects that are found to be seropositive might be justified in case they lack miRNAs in plasma or urine, where otherwise treatment would be discontinued.

Plasma levels of viral miRNAs in seropositive subjects were not different compared to seronegative subjects and both were close to the detection limit, indicating that in healthy subjects only low activity of JCPyV is present in the periphery, independent of their antibody status. In contrast significantly increased urinary viral miRNA levels were found in shedders. In this group of viral shedders, there was also a strong correlation between urinary miRNA levels and anti-VP1 antibody levels or urinary viral load.

Besides the discordance between urine and plasma miRNA levels, also the identity of the detected miRNAs (J1-5p or J1a-5p variant) was different in plasma and urine. The most obvious explanation for these observations would be that two independent viral propagation zones exist in one individual: one in bone marrow cells, blood cells and perhaps also brain cells (Bone-Blood-Brain)and a second one in urinary tract cells. This hypothesis is also supported by other work where sequencing analysis of the VP1 gene and/or non-coding control region (NCCR) in samples obtained from PML patients showed a similar dichotomy between urine on the one hand and plasma and cerebrospinal fluid (CSF) on the other hand. Whether crosstalk between both viral propagation zones exists, is unclear and remains to be investigated.

Increased viral replication in the urinary propagation zone, as determined by increased urinary viral load, is accompanied by an increased level of urinary viral miRNAs. This might at first sight appear in contradiction with the observation that polyomavirus miRNAs, including JCPyV, have been shown to downregulate expression of large T-antigen and to suppress viral replication. This mechanistic model does however not exclude that in subjects with high urinary viral load, viral miRNAs are released at increased levels as a consequence of high viral particle production, a process that requires transcription of the late transcript, which also encodes the miRNAs. Furthermore, several studies have shown that replication and infectivity of mutant viruses lacking the viral miRNAs are not impacted, again indicating that increased levels of viral miRNAs are not per se associated with decreased replication in vivo. On the other hand, these miRNAs can also be produced and released from an infected cell without the need for active viral replication, as is also the case for herpes simples and Epstein-Barr Virus during latent infection. This would in fact also explain why no viral DNA can be detected in plasma of healthy subjects, while viral miRNAs are detectable in a large number of these subjects. Taken together, low levels of circulating viral miRNAs would serve as biomarker for latent JCPyV infection and increased levels might be indicative of increased viral activity. This raises the question whether an increased viral replication in the bone-blood-brain propagation zone, as is the case in PML patients, also is accompanied by an increase in plasma viral miRNAs. Therefore, it would be of great interest to determine miRNA levels in plasma of PML patients, but even more to assess in longitudinal studies whether plasma miRNA levels in MS patients treated with natalizumab increase over time and as such might serve as a monitoring tool for viral reactiviation. Similar studies have been performed for the closely related BKPyV in the context of kidney transplant patients developing polyomavirus-associated nephrophaty (PVAN) and found strongly increased levels of miR-B1-5p in urine from patients with PVAN. Also, a strong correlation appears to exist between BKPyV encoded miRNAs and BKPyV DNA in blood of infected renal transplant patients.

The current invention provides the potential for development of a non-invasive method for early diagnosis of PML (Progressive Multifocal Leukoencephalopathy) by assessing the levels of circulating JCPyV miRNAs in plasma or urine of a human being.

Experimental Part

Healthy Subject Samples

A total of 50 healthy subjects (HSs) were selected for this study. The demographic description of the HS population is presented in Table 1. Plasma samples and urine samples were collected from all these HSs and stored at −80° C. until further processing.

JC Polyomavirus Viral Load Assay Analysis of the urinary viral load was performed as described previously in “Van Loy T, Thys K, Tritsmans L, Stuyver L J (2013) Quasispecies analysis of JC virus DNA present in urine of healthy subjects. PLoS One 8: e70950”. Analysis of the plasma viral load was performed similarly, with the exception that 200 μL plasma was used for DNA extraction.

JC Polyomavirus VP1 Serology Assay

The anti-JCPyV antibody assay was performed as described earlier in “Lagatie O, Van Loy T, Tritsmans L, Stuyver L J (2014) Circulating human microRNAs are not linked to JC polyomavirus serology or urinary viral load in healthy subjects. Virol J 11: 41”. Samples were considered positive if OD values were higher than 2-fold the OD value of the blank sample (i.e. log₂ test/ctrl >1).

Synthetic MicroRNA Molecules and Generation of miRNA Standard Curves

Three RNase-free 5′-phosphorylated miRNA oligoribonucleotides were synthesized (Integrated DNA Technologies) for the validation of the miRNA assays, corresponding to jcv-miR-J1-5p (5′-phospho-UUCUGAGACCUGGGAAAAGCAU-OH-3′) (SEQ ID No:1), jcv-miR-J1a-5p (5′-phospho-UUCUGAGACCUGGGAAGAGCAU-OH-3′) (SEQ ID No:2) and bkv-miR-B1-5p (5′-phospho-AUCUGAGACUUGGGAAGAGCAU-OH-3′) (SEQ ID No:3). Stock solutions of 100 μM synthetic oligonucleotide in RNase-free and DNase-free water were prepared according to the concentrations and sample purity quoted by the manufacturer (based on spectrophotometric analysis). These stock solutions were diluted to a concentration of approximately 3.32 pM, corresponding to 2.10⁵ copies/μL. A total of six serial 10-fold dilutions were prepared, starting from 2.10⁵ copies/μL down to 2 copies/μL and additional no template controls (NTC; zero copies) were examined. Dilution series of each of the synthetic miRNAs were made in RNase-free and DNase-free water.

Analysis of Viral miRNAs

Levels of circulating viral miRNAs were determined by means of quantitative reverse transcriptase PCR (qRT-PCR) with hydrolysis probe based miRNA assays, purchased from Life Technologies. Specific assays were used for jcv-miR-J1-5p, jcv-miR-J1a-5p and bkv-miR-B1-5p. As extraction and reverse transcription efficacy control, assays specific for human miRNAs hsa-miR-26a, hsa-miR-30b and mmu-miR-93 were included in the analysis.

Briefly, RNA was isolated from 200 μL plasma or urine using the miRNeasy kit (Qiagen) according to the manufacturer's instructions. Three μL of total RNA (representing 20% of total RNA extract) or synthetic miRNA solution was reverse transcribed using the pooled RT stem-loop primers (Life Technologies), enabling miRNA specific cDNA synthesis. Subsequently, 2.5 μL of the RT product (representing ⅙ of total RT product) was pre-amplified by means of a 12-cycle PCR reaction with a pool of miRNA specific forward primer and universal reverse primer to increase detection sensitivity. Diluted (1:8) pre-amplified miRNA cDNA was then used as input for a 45-cycle qPCR reaction with miRNA specific hydrolysis probes and primers (Life Technologies). For analysis of the viral miRNAs, 2 μL of input was used. For analysis of the human miRNAs, 2 μL of input was used for urine derived miRNAs and 0.2 μL was used for plasma derived miRNAs. All reactions were performed in duplicate on the LightCycler® 480 instrument (Roche Applied Science). Quantification cycle (Cq) values were calculated using the 2^{nd} Derivative method with a detection cut-off of 40 cycles.

Only samples with quantifiable Cq values for both duplicates were considered positive. Absolute miRNA levels in plasma or urine were calculated based on the standard curves for each specific miRNA assay. Limit of quantification (LOQ) was defined as the miRNA concentration corresponding to a Cq value of 40, based on the standard curve for the specific miRNA. For each sample, average Cq value of the 3 human control miRNAs was calculated and possible outliers were identified using Grubbs' test (using a significance level of 0.05). In case outliers were detected, results of the corresponding viral miRNAs were not included for further analysis.

Statistical Analysis

Differences in relative occurrence of viral miRNAs between groups were assessed using a Fisher's test. Differences between groups were considered statistically significant at P<0.05. Differences in miRNA levels between groups were assessed using a Mann-Whitney test. Differences between groups were considered statistically significant at P<0.05. Correlation between different parameters was analyzed using linear regression. P-value was calculated to determine whether slope was significantly non-zero and goodness of fit was determined using r-value. All statistical analyses were performed using GraphPad Prism v 5.04.

Results

Assay Linearity and Specificity

Plasma or urine levels of JCPyV miRNA were analyzed using stem-loop RT followed by TaqMan PCR analysis. Since the 3p miRNA of JCPyV is identical to the 3p miRNA of BKPyV, only 5p miRNAs were investigated. As we previously identified a variant of the JCV-miR-J1-5p bearing one nucleotide difference compared to the miRNA described in miRBase (designated JCV-miR-J1a-5p), assays specifically designed for both variants were used, as well as an assay detecting the closely related BKPyV 5p miRNA (FIG. 1A). To evaluate the specificity of the assays, standard curves were prepared of each miRNA (JCV-miR-J1-5p, JCV-miR-J1a-5p and BKV-miR-B1-5p) and analyzed using the three specific assays (FIG. 1B-1D). Relative detection efficiency was calculated from the difference of quantification cycle (Cq) between the specific assay and the non-specific assay, using samples containing 2.10⁴ copies/μL of the individual miRNAs (FIG. 1E). Only marginal cross reaction was observed for most combinations, but a substantial cross reaction was observed between JCV-miR-J1a-5p and BKV-miR-B1-5p. Therefore, for every single clinical sample, the contribution of non-specific amplification was calculated and confirmed to be negligible compared to the specific signal of JCV-miR-J1-5p or JCV-miR-J1a-5p. For the BKV-miR-B1-5p assay, however, the calculated contribution of JCV-miR-J1a-5p in most cases was similar to the measured levels, indicating that the BKV-miR-B1-5p assay in most cases actually was detecting JCV-miR-J1a-5p. Consequently, we can conclude that in most samples BKV-miR-B1-5p is absent or present at such low levels that they do not interfere in the interpretation of the JCV-miR-J1-5p and JCV-miR-J1a-5p analyses.

Analysis of JCPyV miRNA Levels in Plasma and Urine

Plasma and urine samples were collected from 50 healthy subjects (HS) and JCPyV VP1 serology and both plasma and urinary viral load was determined . Based on these parameters, subjects were categorized as Ab⁻, Ab⁺VL⁻ or Ab⁺VL⁺ (Table 1). Among these 50 HSs JCPyV VP1 antibody prevalence was 72% (36/50) and 36% (13/36) of this group were shedding virus in their urine, representing 26% (13/50) of the total study population. In the JCPyV VP1 seronegative group, no urinary virus shedding was observed. We found no subjects with detectable JCPyV DNA in plasma, similar to previous observations.

Total RNA, including microRNAs was isolated from both urine and plasma and the level of JCPyV 5p miRNA was quantified in all samples (FIG. 2). The overall detection rate of JCPyV miRNA was 74% (37/50) in plasma and 62% (31/50) in urine (FIG. 3A). Further analysis of the different subgroups shows that JCPyV miRNA was detected in plasma or urine from HS from all subgroups (Ab⁻, Ab⁺VL⁻ or Ab⁺VL⁺) at similar detection rates (P>0.05 between the subgroups for both plasma and urine). In the seropositive group, JCPyV 5p miRNA was detected in plasma of 69% (25/36) of subjects and in urine of 64% (23/36) of subjects. Remarkably, also in the seronegative group, JCPyV 5p miRNA was detected in plasma of 86% (12/14) of subjects and in urine of 57% (8/14) of subjects. These detection rates were not statistically different compared to those in seropositive subjects (P>0.05 both for plasma and urine).

Quantitative analysis of JCPyV 5p miRNA indicated plasma levels of JCPyV 5p miRNA were similar in the three different subgroups (FIG. 3C). In urine, significantly higher levels (P<0.001) were observed in the subgroup shedding JCPyV in their urine compared to the subgroup not shedding JCPyV in their urine (FIG. 3D). No correlation was observed between plasma levels and urine levels of JCPyV 5p miRNA (FIG. 3B). Remarkably, while in plasma higher levels of JCV-miR-J1-5p were detected than JCV-miR-J1a-5p, in urine both variants were detected at similar levels (FIG. 2). Also, the identity of the miRNAs (J1 or J1a variant) was not correlated between urine and plasma. Comparison of JCPyV 5p miRNA levels with JCPyV VP1 serology or urinary viral load showed that, specifically in the subgroup of JCPyV shedders (Ab⁺VL⁺) a good correlation (P<0.005 , r=0.78) exists between miRNA levels and antibody levels, as well as a moderate correlation (P=0.07, r=0.57) with urinary viral load (FIG. 4A-4B). No correlation could be observed between plasma miRNA levels and any other parameter analyzed (FIG. 4C-4D).

### FIGURE LEGENDS

FIG. 1. Validation of the stem-loop RT-PCR miRNA assays. (A) Sequence comparison of the three miRNAs investigated. (B-D) The assay linearity and specificity was evaluated with dilution series of three synthetic miRNAs, miR-J1-5p, miR-J1a-5p and miR-B1-5p. Each dilution series was analyzed using the three miRNA assays. (E) The assay readings of miR-J1-5p by miR-J1-5p assay, miR-J1a-5p by miR-J1a-5p assay, and miR-B1-5p by miR-B1-5p assay at concentration levels of 2.10⁴ copies/μL extract were used as the relative standards (100%) for the analysis of assay cross-reactivity.

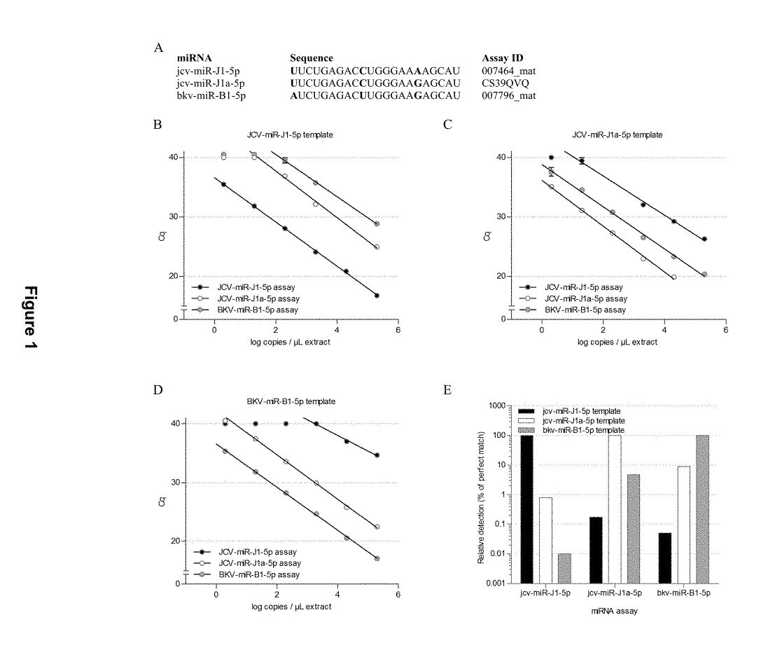

FIG. 2. Individual levels of jcv-miR-J1-5p and jcv-miR-J1a-5p in plasma and urine. Plasma and urine levels (in log copies/mL) of the two JCPyV miRNA variants (jcv-miR-J1-5p and jcv-miR-J1a-5p) in every individual subject.

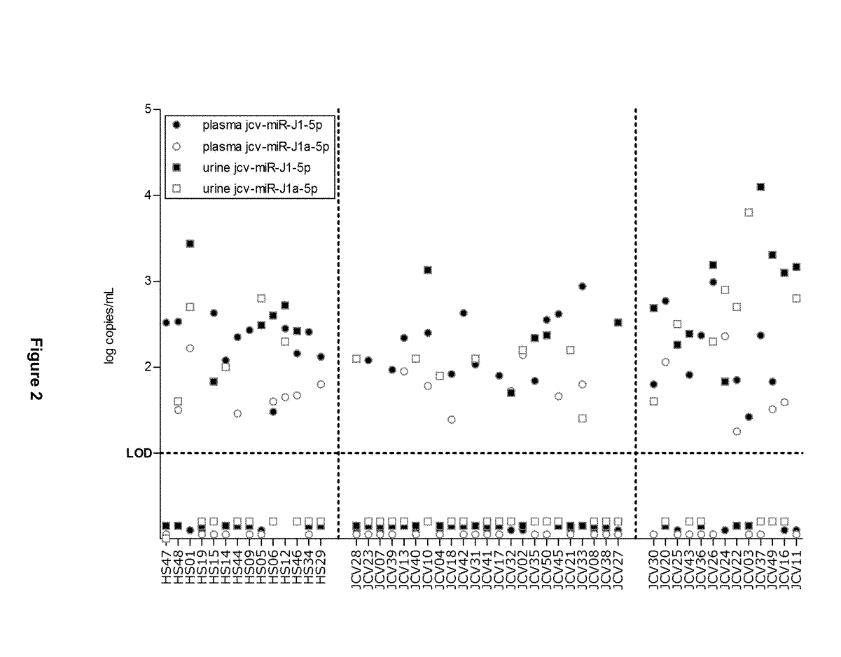

FIG. 3. JCPyV miRNA levels detected in plasma and urine of healthy subjects, categorized based on serology and urinary viral load. (A) Number of subjects with detectable levels of JCPyV miRNAs in plasma and urine in the different groups. (B) Correlation between plasma and urine levels (in log copies/mL) of JCPyV miRNAs. (C) Plasma levels (in log copies/mL) of JCPyV miRNAs in the different groups. In case both variants were detected, the sum of both levels is presented. (D) Urine levels (in log copies/mL) of JCPyV miRNAs in the different groups. In case both variants were detected, the sum of both levels is presented.

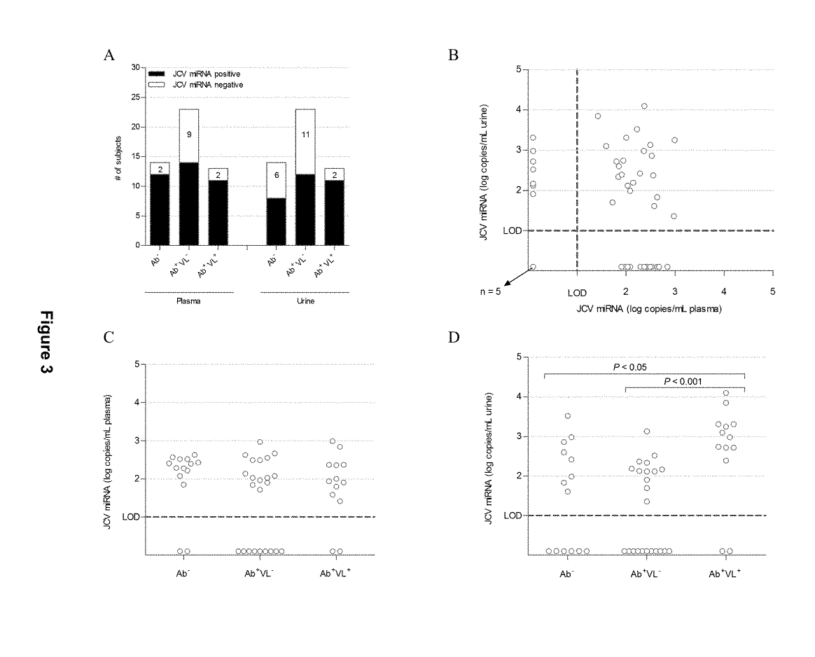

FIG. 4. Viral miRNAs related to other viral parameters. (A) Correlation between JCPyV miRNA levels (in log copies/mL) in urine and JCPyV VP1 serology (in loge test/ctrl). Shedders are indicated in red and P-value and r value are based on this subset only. (B) Correlation between JCPyV miRNA levels (in log copies/mL) in urine and JCPyV urinary viral load (in log copies/mL) in shedders. (C) Correlation between JCPyV miRNA levels (in log copies/mL) in plasma and JCPyV VP1 serology (in loge test/ctrl). Shedders are indicated in red and P-value is based on this subset only. (D) Correlation between JCPyV miRNA levels (in log copies/mL) in plasma and JCPyV urinary viral load (in log copies/mL) in shedders.

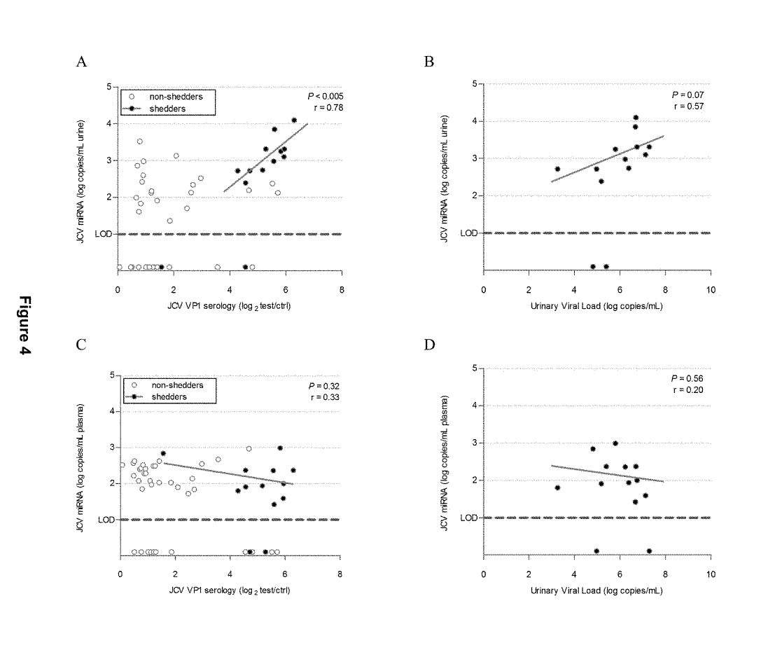

TABLE 1
Overview of subjects investigated

| Variable | Healthy Subjects (n = 50) |
|---|---|
| Gender, n (%) | |
| Male | 22 (44%) |
| Female | 28 (56%) |
| Age, median (Min-Max) | 40.5 (23-59) |
| Race, ethnicity, n (%) | |
| White | 38 (76%) |
| Black | 3 (6%) |
| Asian | 8 (16%) |
| Other/unknown | 1 (2%) |
| VP1 serology, n (%) | |
| Positive | 36 (72%) |
| Negative | 14 (28%) |
| Viruria, n (%) | |
| Positive* | 13 (26%) |
| Negative | 37 (74%) |
| Viremia, n (%) | |
| Positive | 0 (0%) |
| Negative | 50 (100%) |

*All viruric subjects were part of the VP1 seropositive subgroup

1. A method to analyze the presence and/or activity of JCPyV in a subject by quantitatively assessing the levels of circulating JCPyV miRNAs within a sample derived from said subject by detecting said circulating JCPyV miRNAs comprising the steps of:
a. Extracting circulating JCPyV miRNAs from a sample derived from a subject and producing JCPyV miRNA containing miRNA extract;
b. Reverse transcribing said JCPyV miRNA containing miRNA extract and producing JCPyV miRNA cDNA;
c. Amplifying said JCPyV miRNA cDNA and forming a plurality of amplified JCPyV miRNA cDNAs and a plurality of complementary JCPyV miRNA cDNAs; and
d. Detecting the presence and/or quantifying said plurality of amplified JCPyV miRNA cDNAs or said plurality of complementary JCPyV miRNA cDNAs and detecting and/or quantifying said circulating JCPyV miRNAs in said sample.

2. The method according to claim 1 wherein detecting and quantifying said circulating JCPyV miRNAs in said sample identifies a JCPyV-infected subject.

3. The method according to claim 1 or 2, wherein said detection and quantification comprises:
a. Determining an amount of said plurality of amplified JCPyV miRNA cDNAs or said plurality of complementary JCPyV miRNA cDNAs;
b. Determining a level of circulating JCPyV miRNAs within said sample based on said amount; and
c. Comparing said level to a standard control level, wherein said level being higher than said standard control level indicative of said subject developing or having PML.
